(19) United States
(12) Patent Application Publication
(10) Pub. No.: US 2012/0300897 A1
(43) Pub. Date: Nov. 29, 2012

(54) COMPUTED TOMOGRAPHY SYSTEM WITH INTEGRATING AND COUNTING DETECTOR ELEMENTS

(75) Inventors: Thomas Flohr, Uehlfeld (DE); Gabriel Haras, Mucke (DE); Daniel Niederlöhner, Erlangen (DE); Stefan Pflaum, Hirschaid (DE)

(73) Assignee: SIEMENS AKTIENGESELLSCHAFT, Munich (DE)

(21) Appl. No.: 13/478,593

(22) Filed: May 23, 2012

(30) Foreign Application Priority Data
May 24, 2011 (DE) 10 2011 076 358.9

(51) Int. Cl. A61B 6/03

(52) U.S. Cl. 378/9

(57) ABSTRACT

A computed tomography system includes at least two simultaneously operatable sets of detector elements, with at least one first set of integrating detector elements being designed for integrating radiation measurement and at least one second set of counting detector elements being designed to disperse an incident radiation spectrum into at least two energy bins; and includes a computer system for controlling the CT system and at least once capturing the measurement data of the detector elements with a memory and computer programs contained therein. The computer system is provided with programming so that a first mode is provided for operating only the at least one first set of integrating detector elements and a second mode is provided for the additional or sole operation of the at least one second set of counting detector elements.

### PRIORITY STATEMENT

The present application hereby claims priority under 35 U.S.C. §119 to German patent application number DE 10 2011 076 358.9 filed May 24, 2011, the entire contents of which are hereby incorporated herein by reference.

### FIELD

At least one embodiment of the invention generally relates to a computed tomography system with at least two simultaneously operatable sets of detector elements for the simultaneous scanning of an examination object from a plurality of projection angles, with at least one first set of integrating detector elements being designed for integrating radiation measurement, at least one second set of counting detector elements being designed to disperse an incident radiation spectrum into at least two energy bins; and/or a computer system for controlling the CT system and at least once capturing the measurement data of the detector elements with a memory and computer programs contained therein.

### BACKGROUND

CT systems with simultaneously operatable sets of integrating and counting detector elements are generally known. Generally dual-source CT systems are equipped with two detectors disposed with an angle offset, a first detector being provided with a set of integrating detector elements and a second detector being provided with a different set of counting detector elements. It is then possible to scan an examination object, generally a patient, simultaneously using both measuring systems.

### SUMMARY

The inventors have recognized that one problem with such systems is that while approval can be obtained to operate a CT system with integrating detector for routine clinical examinations, CT systems with counting detectors are not given such approval yet or only after very complicated approval procedures. However there is at the same time also a desire at clinical institutions, which also carry out research, to be able to operate a CT system with counting detector systems, in order to be able to utilize the benefits of such detectors in respect of higher spatial resolution and the possibility of measurement with energy resolution for such research.

At least one embodiment of the invention is therefore to find a CT system, which is approved on the one hand for clinical purposes and on the other hand also allows scanning with energy resolution, optionally with better spatial resolution than with integrating detectors, without the need for approval for such a scanning procedure for medical purposes.

In at least one embodiment, the inventors propose improving a computed tomography system with at least two simultaneously operatable sets of detector elements for the simultaneous scanning of an examination object from a plurality of projection angles, with at least one first set of integrating detector elements being designed for integrating radiation measurement, at least one second set of counting detector elements being designed to disperse an incident radiation spectrum into at least two energy bins, and with a computer system for controlling the CT system and at least once capturing the measurement data of the detector elements with a memory and computer programs contained therein. The improvement is achieved in that the computer system is provided with programming so that a first mode is provided for operating only the at least one first set of integrating detector elements and a second mode is provided for the additional or sole operation of the at least one second set of counting detector elements.

Advantageous developments of the invention are the subject matter of subordinate claims.

### BRIEF DESCRIPTION OF THE DRAWINGS

The invention is described in more detail below with the aid of the figures, in which only the features required to understand the invention are shown. The following reference characters are used: 1: CT system; 2: first x-ray tube; 3: first detector; 4: second x-ray tube; 5: second detector; 6: gantry housing; 6.1: gantry; 7: patient; 8: patient couch; 9: system axis; 10: computer system; 10.1, 10.2: memory; 10.3: slot; I: integrating detector elements; Prg (I): first set of computer programs; Prg (II): second set of computer programs; Z: counting detector elements.

In detail:

FIG. 1: shows a CT system;

FIG. 2: shows a gantry with two detectors disposed with an angle offset, each having integrating or counting detector elements in clinical operating mode;

FIG. 3: shows a gantry with two detectors disposed with an angle offset, each having integrating or counting detector elements in research and development operating mode;

FIG. 4: shows a plan view of a detector with both integrating detector elements and counting detector elements grouped in rows;

FIG. 5: shows a plan view of a detector with both integrating detector elements and counting detector elements grouped in lines;

FIG. 6: shows a plan view of a detector with a number of directly adjacent lines of integrating detector elements and a number of adjoining directly adjacent lines of counting detector elements;

FIG. 7: shows a plan view of a detector with a honeycomb arrangement of octagonal integrating detector elements with counting detector elements in the gaps.

It should be noted that these Figures are intended to illustrate the general characteristics of methods, structure and/or materials utilized in certain example embodiments and to supplement the written description provided below. These drawings are not, however, to scale and may not precisely reflect the precise structural or performance characteristics of any given embodiment, and should not be interpreted as defining or limiting the range of values or properties encompassed by example embodiments. The use of similar or identical reference numbers in the various drawings is intended to indicate the presence of a similar or identical element or feature.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Methods discussed below, some of which are illustrated by the flow charts, may be implemented by hardware, software, firmware, middleware, microcode, hardware description languages, or any combination thereof. When implemented in software, firmware, middleware or microcode, the program code or code segments to perform the necessary tasks will be stored in a machine or computer readable medium such as a storage medium or non-transitory computer readable medium. A processor(s) will perform the necessary tasks.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the following description, illustrative embodiments may be described with reference to acts and symbolic representations of operations (e.g., in the form of flowcharts) that may be implemented as program modules or functional processes include routines, programs, objects, components, data structures, etc., that perform particular tasks or implement particular abstract data types and may be implemented using existing hardware at existing network elements. Such existing hardware may include one or more Central Processing Units (CPUs), digital signal processors (DSPs), application-specific-integrated-circuits, field programmable gate arrays (FPGAs) computers or the like.

Note also that the software implemented aspects of the example embodiments may be typically encoded on some form of program storage medium or implemented over some type of transmission medium. The program storage medium (e.g., non-transitory storage medium) may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The example embodiments not limited by these aspects of any given implementation.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

At least one embodiment of the invention is therefore to find a CT system, which is approved on the one hand for clinical purposes and on the other hand also allows scanning with energy resolution, optionally with better spatial resolution than with integrating detectors, without the need for approval for such a scanning procedure for medical purposes.

The inventors have identified the following:

Computed tomography (CT) devices used in medicine are currently equipped with integrating scintillation detectors. In these, the incident x-ray beams are first converted to visible light in a two-stage process, the visible light then being detected by downstream photodiodes and being converted to electrical signals. Examples of corresponding scintillators are gadolinium oxide and gadolinium oxide sulfide. Such scintillation detectors have a very broad dynamic range and can process the minimum and maximum x-ray flux densities used in medical computed tomography without any problems. On the other hand their spatial resolution is limited, since for mechanical reasons the detector pixels cannot be reduced in any manner for mechanical and optical separation due to inactive dead zones between the pixels. Furthermore integrating scintillation detectors do not provide spectral information, so that x-ray absorption differences due to material characteristics for different energies of the x-ray spectrum cannot be detected directly. Also the contrast-noise ratio of the detected signals from integrating detectors is not optimal, since the low-energy quanta, which carry most contrast information, are only weighted to a small degree in the integrating detector based on their low energy, thereby reducing the contrast of certain materials, e.g. white and gray brain mass.

In contrast there are counting detectors, in which the incident x-ray quanta are converted to electrical signals and counted in a direct process. Examples of corresponding detector materials are cadmium telluride or cadmium zinc telluride. Counting detectors can be very finely structured in respect of surface, since the pixels do not have to be mechanically separated and there are therefore no dead zones. This allows much higher spatial resolution than with conventional integrating scintillation detectors. Also the incident x-ray quanta can be detected in different energy bands for spectral resolution, with the result that x-ray absorption differences due to material characteristics for different energies can be detected with a single measurement. Because it is possible to weight the contributions to the overall signal as a function of energy, it is also possible to improve object contrast and therefore the contrast-noise ratio compared with integrating scintillation detectors.

However counting detectors only have a limited dynamic range due to the detector materials used, in which a maximum x-ray flux density cannot be exceeded, and this is not high enough for unlimited use in a medical CT system. Also counting detectors are subject to high signal drift as a function of the history of a preceding irradiation in respect of applied dose, dose output and recovery phases.

The above variants relate in each instance to separate detectors with different types of detector elements, in other words with just integrating or counting detector elements. However the scope of the invention also includes hybrid detectors, which comprise both integrating and counting detector elements.

In order to be able to utilize the advantages of such counting detector systems, a considerable amount of development and research work is still required and this is frequently carried out at institutions which also carry out routine medical examinations at the same time. However there is also the need for CT systems used for clinical purposes to be approved, which is only feasible with reasonable outlay at present for conventional detector systems with integrating detectors.

The inventors have identified that, to avoid double purchasing costs for a conventional CT system on the one hand and a CT system that is suitable for research purposes, which also allows scanning with counting detector elements, on the other hand, it is possible to operate a single CT system with two fundamentally different operating modes, a first mode being configured solely for routine clinical operation and only allowing scanning with integrating detector elements. If such an operating mode is used with a dual-source CT system with a first emitter/detector system with only integrating detector elements and a second emitter/detector system with only counting detector elements, in a first operating mode the first emitter/detector system alone can be activated and the second emitter/detector system can be deactivated. It is relatively easy to obtain operating approval for clinical purposes for such a configuration.

On the other hand the CT system can also be started in or switched to a second operating mode completely independently thereof for research purposes, said second operating mode allowing both emitter/detector systems to be activated. Approval for clinical operation is not required here. Generally this embodiment of a CT system with two fundamentally independent and separate operating modes allows both approved clinical operation and extended research operation on a single device by purchasing such a system.

According to these inventive ideas, in at least one embodiment, the inventors propose improving a computed tomography system with at least two simultaneously operatable sets of detector elements for the simultaneous scanning of an examination object from a plurality of projection angles, with at least one first set of integrating detector elements being designed for integrating radiation measurement, at least one second set of counting detector elements being designed to disperse an incident radiation spectrum into at least two energy bins, and with a computer system for controlling the CT system and at least once capturing the measurement data of the detector elements with a memory and computer programs contained therein. The improvement is achieved in that the computer system is provided with programming so that a first mode is provided for operating only the at least one first set of integrating detector elements and a second mode is provided for the additional or sole operation of the at least one second set of counting detector elements.

It is favorable here if a first complete software set is present to operate the first mode and a second complete software set is present to operate the second mode. This allows safe clinical operation to be achieved with a first tested software set, while in the second mode, for example in the field of research activities or material tests on non-living examination objects, it is possible to access the software without any problems.

One possibility for providing such different operating modes is to provide a boot manager to start the operating software—in other words the operating system—said boot manager being embodied so that the selection for operating the first mode or for operating the second mode can be made in a selection menu of the boot manager.

Alternatively to this end the memory of the computer system can also be configured as a removable storage medium, on which only one complete software set for a single operating mode is stored in each instance. Therefore by replacing a hard disk—for example HDD=Hard Disk Drive or SSD=Solid State Drive—a memory card or other removable storage medium, on which the complete software system for the respective operating mode is stored, the CT system can be set to the corresponding mode.

Additionally it can assist operating safety, if a visual and/or acoustic warning display is present, which is automatically activated during operation of a selected operating mode. Thus for example in non-clinical operating mode a corresponding warning display can warn of this. Alternatively operation of the patient couch associated with the system or movement of the patient couch to a specific rest position can be triggered as a warning signal. Similarly for example a specific background color or specific background image can appear as a warning on the screen on which operation of the CT system is controlled or a specific color can be selected for the operating menu.

In a further variant of an embodiment of the inventive computed tomography system, to ensure that only approved operating modes are used for clinical purposes, it is necessary to input a password or proof of authorization at the start of at least one selected operating mode.

In a further embodiment of the computed tomography system it is proposed on the one hand that the at least one first set of integrating detector elements and the at least one second set of counting detector elements are disposed respectively on physically different detectors. In principle this requires at least two emitter/detector systems, it being possible only to activate the one with the integrating detector during clinical operation.

Alternatively however at least one hybridized detector can be used, so that the at least one first set of integrating detector elements and the at least one second set of counting detector elements are disposed in a single detector. The integrating detector elements of the at least one first set and the counting detector elements of the at least one second set can be grouped in lines or rows here or can even be distributed in a checkered manner in a detector. A further alternative is a honeycomb arrangement of octagonal integrating detector elements, with square counting detector elements being positioned in the square gaps that form. Finally the integrating detector elements of the at least one first set can also be disposed in a number of directly adjacent lines and the counting detector elements of the at least one second set can be disposed in a number of adjoining lines on at least one or a single detector.

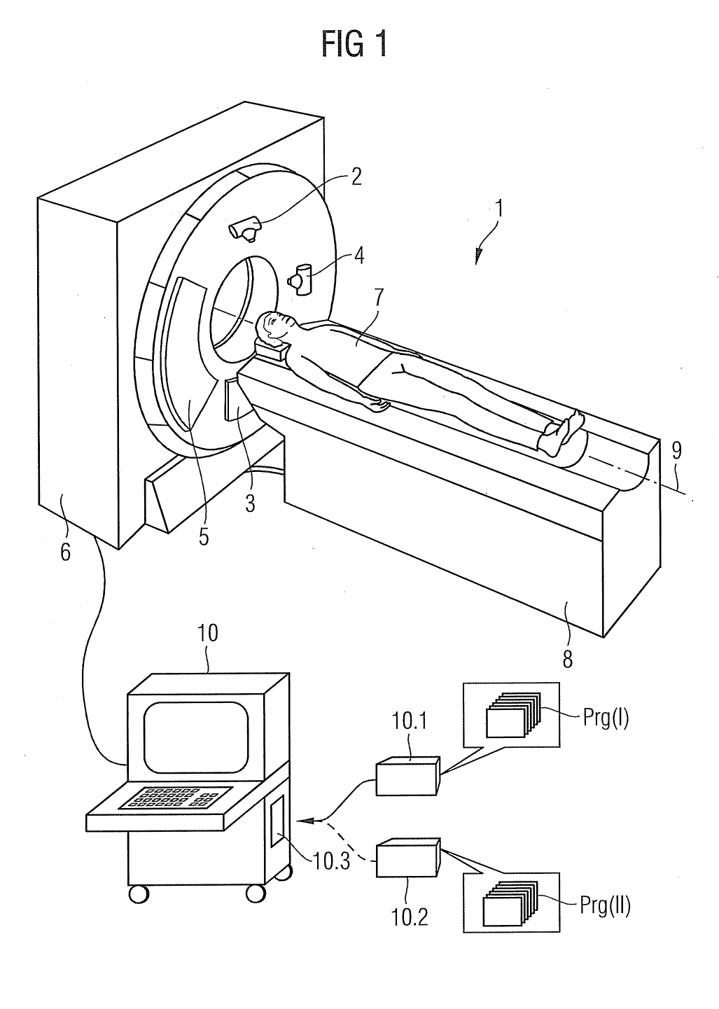

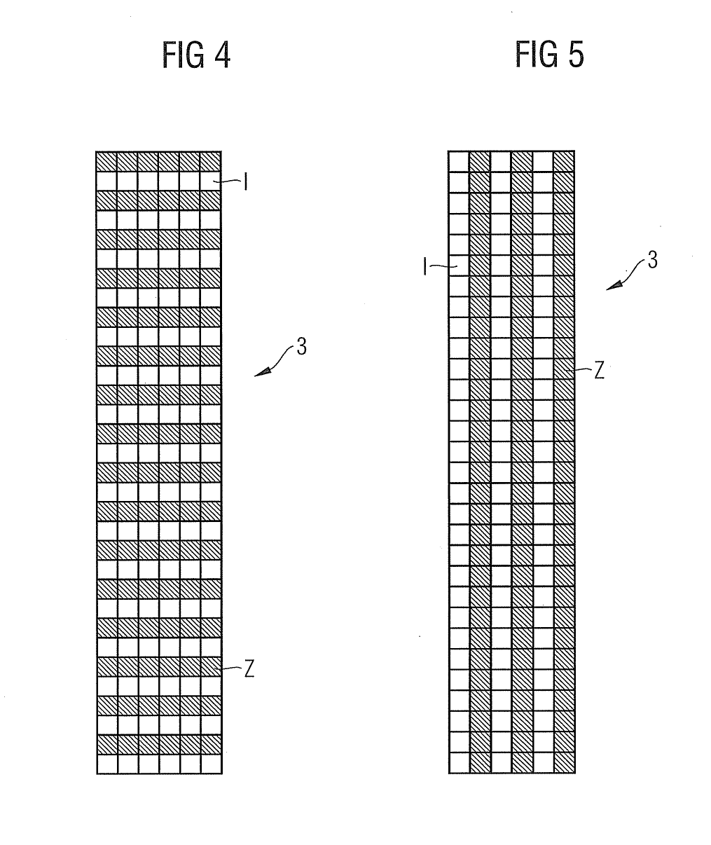

FIG. 1 shows an example CT system 1 with two emitter/detector systems on a gantry (not shown in detail) in a gantry housing 6. The two emitter/detector systems, consisting of a first x-ray tube 2 with an opposing detector 3 assigned to the first x-ray tube on the one hand and a second x-ray tube 4 with an opposing detector 5 assigned to the second x-ray tube on the other hand. Both emitter/detector systems are disposed with an angle offset in a rotational plane on the gantry. According to the invention the two detectors 3 and 5 can be equipped respectively with detector elements with a different function so that one detector is equipped with counting detector elements and the other detector with integrating detector elements. Alternatively it is also possible for the detectors or at least one detector to be hybridized, in that some integrating detector elements and some counting detector elements are incorporated in one detector. Reference is also made to FIGS. 4 to 7 for example distributions of the differently operating detector elements.

Both emitter/detector systems cover a measurement field located in the central round aperture. The patient 7 can be moved through this measurement field along the system axis 9 with the aid of the patient couch 8. It is possible in principle here to perform both a spiral scan and a sequence scan.

Control of the CT systems 1 and evaluation of the scan of the patient 7 are performed by the computer system 10 connected thereto, comprising at least one memory 10.1 and 10.2, in which complete sets of computer programs Prg(I) or Prg(II) are stored. In the embodiment shown here the memories 10.1 and 10.2 are removable storage media, each of which can be inserted into a slot 10.3, thereby defining the different modes in which the CT system is operated.

A complete set of computer programs here is understood to be the sum of all programs necessary to operate the CT system, in other words at least the operating system including all the control software required for operation. This can be supplemented by measurement data acquisition software and optionally also evaluation software. Naturally it also comes within the scope of the invention if a removable storage medium is not only understood to mean a single physical removable storage medium (e.g. FDD, HDD, SSD, SD card, CF card) but a set of removable storage media. A set of removable storage media can thus consist of a number of removable storage media, which are combined for data security into a RAID (=Redundant Array of Independent Disks). A combination of a single memory for the rudimentary operating system and one or more RAIDs for the remainder of the software also comes within the scope of the invention. It is however important here that—as soon as the CT system starts to operate—it is not possible simply to switch between the individual operating modes but rather that a system restart is necessary for this purpose.

According to an embodiment of the invention all further programs and program parts are contained or stored therein, being configured so that they perform the different embodiments of the inventive method during operation of the system.

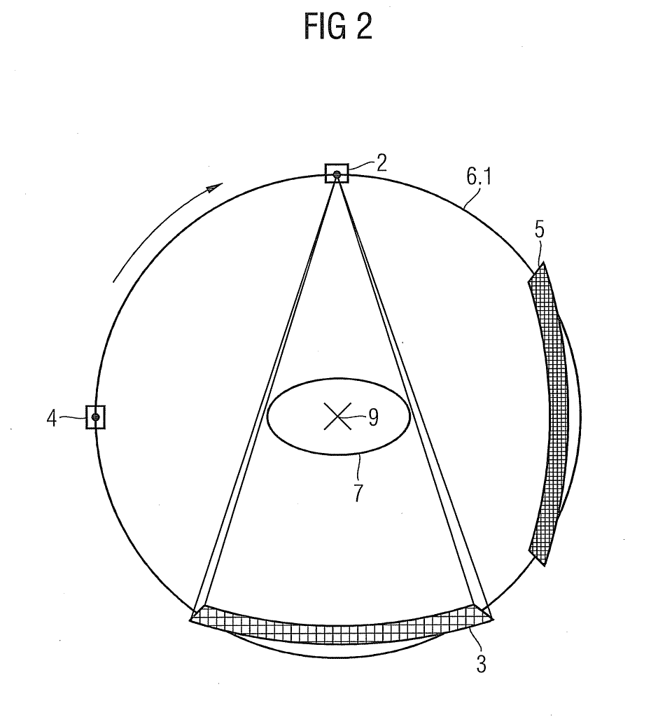

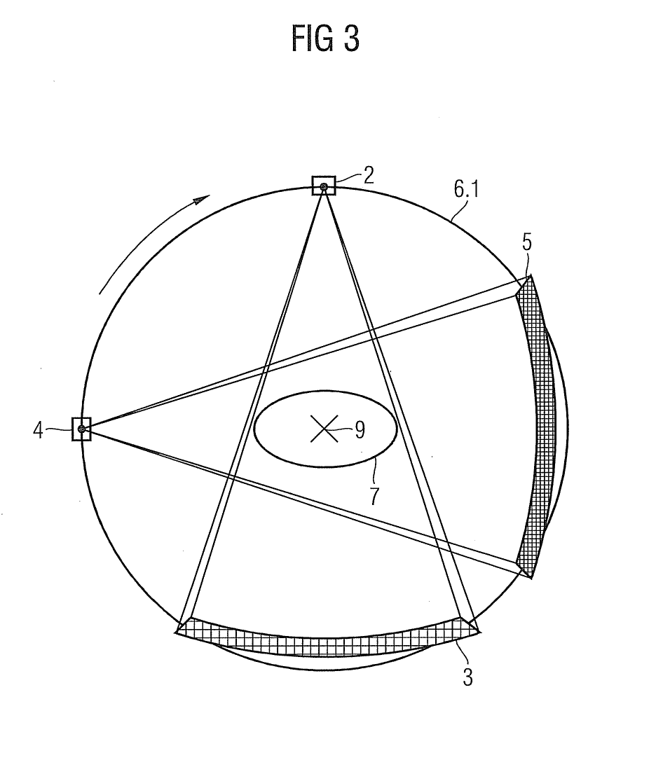

One variant of the CT system is shown schematically as a cross section through the gantry 6.1 in FIGS. 2 and 3 with different operating modes. Two emitter/detector systems are shown in this embodiment, the first emitter/detector system consisting of the x-ray tube (emitter) 2 and an opposing detector 3 being equipped only with integrating detector elements—indicated by the larger detector grid. The second emitter/detector system consists of the x-ray tube (emitter) 4 and a further opposing detector 5 only with counting detector elements—indicated by the smaller detector grid. The patient 7 and the system axis 9 can be seen in the region of the beam bundle between emitter and detector.

In FIG. 2 the CT system is operated in a first mode, which can be approved simply for the clinical application, since during operation only the first emitter/detector system 2, 3 with the conventional detector is active. To illustrate the deactivated state of the second emitter/detector system 4, 5, no beam bundle is shown. FIG. 3 in contrast shows an operating mode intended for research and development, in which both emitter/detector systems are activated—as shown by the two beam bundles marked in.

It should be noted that instead of the detector system with only counting detector elements, hybrid detector systems consisting of a combination of counting and integrating detector elements can also be used in the variant described above.

Examples of different mixed arrangements of integrating and counting detector elements in one, optionally also the single, detector of a CT system are shown in FIGS. 4 to 7.

FIG. 4 shows an embodiment of a hybrid detector with integrating—not shown hatched—and counting—shown hatched—detector elements I, Z disposed in alternate rows. Such an arrangement is suitable in particular for circular scans, since with these every integrating detector element I is followed by a counting detector element Z as they circle and congruent beams can very easily be achieved through the scanned measurement object, being scanned by both types of detector element I, Z, to compare them and optionally be able to correct the measurement data of the counting detector elements Z.

The distribution illustrated in FIG. 5 appears particularly advantageous for use in spiral scans. Integrating—not shown hatched—and counting—shown hatched—detector elements I, Z are disposed here in alternate lines. Since a spiral scan involves an advance in the direction of the system axis, both types of detector element I, Z again pass identical or at least almost identical beams through the measurement object, in order to be able to compare the associated measurement results and optionally to be able to correct the measurement data of the counting detector elements Z. If beams that do not overlap precisely are found from both sets of measurement data, it is possible, by corresponding interpolation operations known per se, to produce interpolated measurement data for congruent beams and optionally also opposing beams.

It should be noted that it also comes within the scope of the invention to use smaller detector elements Z in respect of their projection surface for the counting detectors than for the integrating detector elements I. For example every surface of a counting detector element Z shown here can also be divided up a number of times—for example into 2×2, 3×3 or 4×4 separately readable subsurfaces—so that a much higher spatial resolution results.

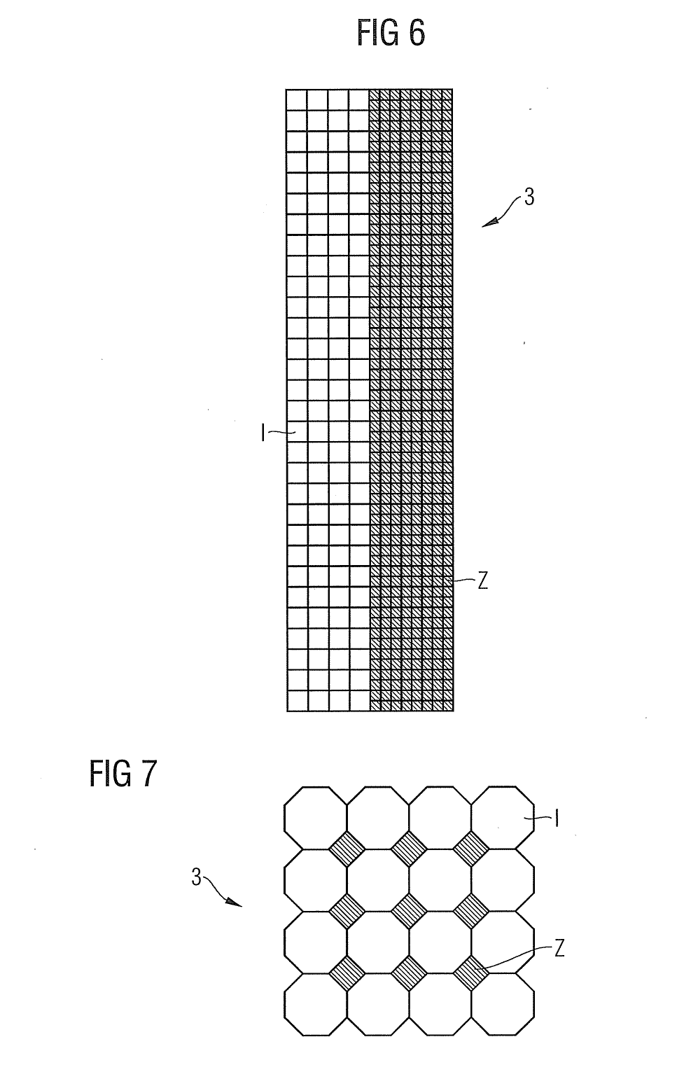

FIG. 6 shows a plan view of a detector with a number of directly adjacent lines of integrating detector elements I and a number of adjoining directly adjacent lines of counting detector elements Z. The integrating detector elements I are again shown not hatched and the counting detector elements Z are shown hatched. The counting detector elements Z are also only half the size in respect of their extension; in other words the spatial measurement resolution of the counting detector elements Z is double that of the integrating detector elements I.

FIG. 7 shows a further advantageous variant of a hybrid arrangement of counting and integrating detector elements Z, I. Here the integrating—shown not hatched—detector elements I are arranged in a honeycomb of octagonal surfaces, with much smaller counting detector elements Z being positioned in the resulting square gaps in the surface, thereby not only allowing the energy resolution of the recorded spectrum but also defining finer measurement beams.

If only hybrid detectors are used in a CT system, in a clinical operating mode all the counting detector elements Z can be deactivated and in a non-clinical operating mode both the counting and the integrating detector elements Z, I or just the counting detector elements Z can be activated, it not being necessary to deactivate an emitter in either of the operating modes. The possible variations here are based on the rules for approval for clinical operation.

Generally therefore an embodiment of the invention describes a computed tomography system with at least two simultaneously operatable sets of integrating detector elements with energy resolution, wherein a computer system is provided to capture measurement data of the detector elements with a memory and computer programs contained therein, so that a first mode is available for operating only the at least one first set of integrating detector elements and a second mode for the additional or sole operation of the at least one second set of counting detector elements.

Although the invention has been illustrated and described in detail using the preferred example embodiment, the invention is not limited by the disclosed examples and other variations can be derived therefrom by the person skilled in the art without departing from the scope of protection of the invention.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of, the aforementioned methods may be embodied in the form of a system or device, including, but not limited to, any of the structure for performing the methodology illustrated in the drawings.

Even further, any of the aforementioned methods may be embodied in the form of a program. The program may be stored on a tangible computer readable medium and is adapted to perform any one of the aforementioned methods when run on a computer device (a device including a processor). Thus, the tangible storage medium or tangible computer readable medium, is adapted to store information and is adapted to interact with a data processing facility or computer device to execute the program of any of the above mentioned embodiments and/or to perform the method of any of the above mentioned embodiments.

The tangible computer readable medium or tangible storage medium may be a built-in medium installed inside a computer device main body or a removable tangible medium arranged so that it can be separated from the computer device main body. Examples of the built-in tangible medium include, but are not limited to, rewriteable non-volatile memories, such as ROMs and flash memories, and hard disks. Examples of the removable tangible medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A computed tomography system, comprising:
at least two simultaneously operatable sets of detector elements for the simultaneous scanning of an examination object from a plurality of projection angles, at least one first set of integrating detector elements being designed for integrating radiation measurement and at least one second set of counting detector elements being designed to disperse an incident radiation spectrum into at least two energy bins; and
a computer system for controlling the CT system and at least once capturing the measurement data of the detector elements with a memory and computer programs contained therein, the computer system being provided with programming so that a first mode is provided for operating only the at least one first set of integrating detector elements and a second mode is provided for the additional or sole operation of the at least one second set of counting detector elements.

2. The computed tomography system of claim 1, wherein a first complete software set is present to operate the first mode and a second complete software set is present to operate the second mode.

3. The computed tomography system of claim 2, wherein a boot manager is provided to start the operating software, being embodied so that the selection for operating the first mode or for operating the second mode can be made in a selection menu of the boot manager.

4. The computed tomography system of claim 2, wherein the memory of the computer system is a removable storage medium, on which only one complete software set for a single operating mode is stored in each instance.

5. The computed tomography system of claim 1, wherein at least one of a visual and acoustic warning display is present, which is automatically activated during operation of a selected operating mode.

6. The computed tomography system of claim 1, wherein input of a password or proof of authorization is needed at the start of at least one selected operating mode.

7. The computed tomography system of claim 1, wherein the at least one first set of integrating detector elements and the at least one second set of counting detector elements are disposed respectively on physically different detectors.

8. The computed tomography system of claim 1, wherein the at least one first set of integrating detector elements and the at least one second set of counting detector elements are disposed in one detector.

9. The computed tomography system of claim 1, wherein the integrating detector elements of the at least one first set and the counting detector elements of the at least one second set are grouped in lines in the one detector.

10. The computed tomography system of claim 1, wherein the integrating detector elements of the at least one first set and the counting detector elements of the at least one second set are grouped in rows in the one detector.

11. The computed tomography system of claim 1, wherein the integrating detector elements of the at least one first set and the counting detector elements of the at least one second set are grouped in a checkered manner in the one detector.

12. The computed tomography system of claim 1, wherein the integrating detector elements of the at least one first set are disposed in a number of directly adjacent lines and the counting detector elements of the at least one second set are disposed in a number of adjoining lines on at least one detector.

13. The computed tomography system of claim 2, wherein the at least one first set of integrating detector elements and the at least one second set of counting detector elements are disposed respectively on physically different detectors.

14. The computed tomography system of claim 2, wherein the at least one first set of integrating detector elements and the at least one second set of counting detector elements are disposed in one detector.

15. The computed tomography system of claim 2, wherein the integrating detector elements of the at least one first set and the counting detector elements of the at least one second set are grouped in lines in the one detector.

16. The computed tomography system of claim 2, wherein the integrating detector elements of the at least one first set and the counting detector elements of the at least one second set are grouped in rows in the one detector.

17. The computed tomography system of claim 2, wherein the integrating detector elements of the at least one first set and the counting detector elements of the at least one second set are grouped in a checkered manner in the one detector.

18. The computed tomography system of claim 2, wherein the integrating detector elements of the at least one first set are disposed in a number of directly adjacent lines and the counting detector elements of the at least one second set are disposed in a number of adjoining lines on at least one detector.
